(19) United States
(12) Patent Application Publication
(10) Pub. No.: US 2014/0105967 A1
(43) Pub. Date: Apr. 17, 2014

(54) STABILIZED COMPOSITION AND METHOD FOR DERMATOLOGICAL TREATMENT

(71) Applicant: Ferndale IP, Inc., Ferndale, MI (US)

(72) Inventor: Pravin M. Patel, West Bloomfield, MI (US)

(73) Assignee: FERNDALE IP, INC., Ferndale, MI (US)

(21) Appl. No.: 14/107,515

(22) Filed: Dec. 16, 2013

Related U.S. Application Data
(63) Continuation of application No. 13/035,403, filed on Feb. 25, 2011.
Application No. 13/035,403 is a continuation-in-part of application No. 12/371,783, filed on Feb. 16, 2009, now abandoned.
Application No. 12/371,783 is a continuation of application No. 10/425,359, filed on Apr. 29, 2003, now abandoned.
(60) Provisional application No. 60/377,002, filed on Apr. 30, 2002.

(51) Int. Cl. A61K 9/127; A61K 31/7056; A61K 31/203

(52) U.S. Cl. CPC A61K 9/127; A61K 31/203; A61K 31/7056
USPC 424/450; 514/24

(57) ABSTRACT

A stabilized, topical composition for the treatment of acne and other dermatological conditions comprises a liposomal formulation of a retinoid and an antibiotic in which the retinoid is disposed in the lipid phase of the formulation, and the antibiotic is disposed in the aqueous phase so as to be isolated from the retinoid. Lincosamides, such as clindamycin, are one group of antibiotics which may be used in the composition. Tretinoin is one preferred retinoid. Also disclosed are methods for making the compositions and methods for using the composition.

### REFERENCE TO RELATED APPLICATIONS

This patent application is a continuation of U.S. patent application Ser. No. 13/035,403, filed Feb. 25, 2011, which is a continuation-in-part of U.S. patent application Ser. No. 12/371,783, filed Feb. 16, 2009, now abandoned, which is a continuation of U.S. patent application Ser. No. 10/425,359, filed Apr. 29, 2003, now abandoned, which claims priority from U.S. Provisional Patent Application Ser. No. 60/377,002, filed Apr. 30, 2002. The entire content of each application is incorporated herein by reference.

### FIELD OF THE INVENTION

This invention relates generally to the treatment of skin conditions such as acne. More specifically, the invention relates to storage-stable, dermatological compositions based upon liposomal formulations of retinoids and antibiotics, and to their use for the treatment of skin conditions such as acne.

### BACKGROUND OF THE INVENTION

Acne is a dermatological disorder which occurs when inflamed sebaceous glands become blocked with sebum, skin cells and bacteria. Lesions occur in more superficial forms as open or closed comedones, as well as in deeper varieties such as nodules and cysts. Acne tends to appear at the onset of puberty and persists into early adulthood. One reason for the association of acne with puberty is that sebum levels are under hormonal control. While not usually physically disabling, acne can be particularly disturbing for cosmetic reasons to those affected. In addition, untreated or inappropriately treated acne can result in permanent, disfiguring scarring.

One approach to the treatment of acne relies upon the systemic administration of antibiotics and/or isotretinoin. Antibiotics such as tetracycline have been given orally to target bacteria associated with the skin such as Propionibacterium acnes. However, systemic administration has the drawback of affecting all areas of the body and disrupting endogenous flora not involved in acne, and producing side effects such as nausea and diarrhea. Isotretinoin is a highly effective systemic anti-acne agent which is given in cases of severe acne. However, isotretinoin has significant side effects and is contraindicated for women of childbearing age. Consequently, such systemic therapies are of somewhat limited utility; therefore, there is a need for effective topical therapies.

A variety of topical acne therapies have been proposed and utilized. These therapies are usually fairly adequate for relatively mild cases of acne; however, they are often inadequate for relatively severe cases. Prior art topical therapies generally relied upon various combinations of antibiotics and/or retinoids together with organic peroxides such as benzoyl peroxide and anti-inflammatory compositions such as corticosteroids. The utility of prior art compositions has been limited by a number of factors.

For reasons of patient compliance, ease of dispensing and accuracy of dosing, it is usually desirable that combination therapies used for the topical treatment of acne employ a single preparation containing two or more active ingredients. Oftentimes materials employed in such compositions are not mutually compatible in a common carrier or vehicle; hence problems of oxidation, phase separation or chemical degradation may occur and can affect the shelf life and efficacy of such materials. For example, it has been found that compositions containing retinoids and antibiotics are not storage stable over moderately long periods of time. In order to avoid these stability problems, compositions may be prepared shortly before use, but doing so is commercially and logistically impractical. In other instances, combination therapies may be employed wherein separate topical compositions are applied to a patient or combinations of oral and topical therapy are implemented. As noted above, patient compliance is an issue in therapies requiring the separate application of discrete topical formulations, and oral administration of various acne therapeutics can be limited by side effects. While the prior art has prepared various compositions containing antibiotics and retinoids, and while some of these compositions have included liposomal or other emulsified vehicles, the prior art has never prepared topical compositions in which antibiotics and retinoids have been isolated from one another nor has the prior art recognized any advantages of doing so.

As will be explained in greater detail hereinbelow, the present invention is directed to stabilized, topical compositions for the treatment of dermatological conditions. While the compositions of the present invention have significant and primary utility in the treatment of acne, they can also be employed for other dermatological conditions including conditions in which microbial infection is a factor, as well as noninfective conditions such as rosacea. The compositions of the present invention are based upon a liposomal structure in which a discontinuous, lipid phase material is dispersed in a continuous, aqueous phase material, which is most preferably a thickened or gelled aqueous phase material. The liposomal nature of the preparations of the present invention allows for the isolation of mutually incompatible active ingredients such as retinoids and antibiotics thereby greatly enhancing the long-term storage stability of these preparations.

### BRIEF DESCRIPTION OF THE INVENTION

There is disclosed herein a storage-stable, topical composition for the treatment of skin conditions. The composition includes a liposomal vehicle comprising a discontinuous, lipid phase dispersed in a continuous, aqueous phase. An antibiotic is disposed in the aqueous phase and a retinoid is disposed in the lipid phase. The retinoid is, in particular embodiments, tretinoin. The antibiotic is, in particular compositions, a lincosamide antibiotic, with clindamycin being one particular lincosamide having utility in this invention.

In particular embodiments, the retinoid component is present in a range of 0.01-1.0% by weight. In particular instances, the retinoid is present in an amount of 0.01-0.1% by weight, and in some specific compositions the retinoid is present in an amount of 0.025-0.05% by weight. The composition may include further ingredients such as emollients, antioxidants, surfactants, thickeners, gelling agents, pH control agents, solvents, carriers, dispersion agents, fragrances and colors.

Also disclosed herein is a method for using the composition for treating acne and other skin conditions, as well as methods for the manufacture of the composition.

### DETAILED DESCRIPTION OF THE INVENTION

The present invention provides stabilized compositions and methods for the topical treatment of acne. The compositions are formulated as a liposomal preparation having an aqueous or continuous phase containing an antibiotic as well as a lipid phase which contains a retinoid. Isolation of the antibiotic from the retinoid enhances the storage stability of the compositions. The compositions may also include a carrier or dispersion medium, a solvent, an emollient, an antioxidant, a surfactant, a thickener or gelling agent and a pH stabilizer, as well as coloring agents, fragrances and like ancillary ingredients.

One specific composition includes tretinoin as a retinoid, preferably at a concentration ranging from 0.01-1.0% w/w. In another instance, tretinoin is included at a concentration ranging from 0.01-0.1% w/w. In one specific preparation, tretinoin is present at 0.025-0.05% w/w. In another specific composition, tretinoin is present in the range of 0.025-0.035% w/w.

An antibiotic of the lincosamide class is one specific type of antibiotic used in a composition according to the invention, and clindamycin is one particular lincosamide having utility in this invention. In particular, clindamycin phosphate is found to be especially effective and is one preferred ingredient of the inventive composition. Clindamycin phosphate is included at a concentration ranging from 0.5-10% w/w (calculated as clindamycin base), but is, in particular instances, included at a concentration ranging from 0.75-2.0% w/w (calculated as clindamycin base). Clindamycin may also be included as a free base or other suitable salt, including for example, clindamycin HCl.

In the broadest sense, the compositions of the present invention are based upon liposomal formulations comprising a discontinuous, lipid phase which includes the retinoid, and which is dispersed in a continuous, aqueous phase, which includes the antibiotic. The multiphase nature of the material of the present invention has been found to greatly enhance the stability and therapeutic efficacy of the compositions.

As is known in the art, liposomal materials comprise vesicles of the lipid phase dispersed throughout the aqueous phase. Typically, the walls of the vesicles are comprised of a phospholipid material such as phosphatidyl choline. One such material is available from the Rhone-Poulenc Rorer Phospholipid GmbH under the designation Phospholipon®. As is known in the art, the lipid phase may include stabilizing materials such as cholesterol as well as surfactants, antioxidants and the like. The aqueous phase of the material may be based upon an aqueous solution of the antibiotic. However, in specifically preferred embodiments, this phase is thickened or gelled with art known, compatible, thickening agents such as Carbopol® water-soluble, acrylic resins. Compositions in accord with the present invention may also include ancillary ingredients such as emollients, antioxidants, surfactants, pH control agents, additional solvents, additional carriers, dispersion agents, coloring agents, fragrances and the like. These ancillary ingredients may be added to either or both of the phases. Carriers and dispersion media are known and will be recognized as suitable by one of ordinary skill in the art. Propylene glycol is one particularly preferred carrier which may be used in a topical treatment composition of the present invention. Solvents useful in dissolving tretinoin or clindamycin independently for use in preparing compositions according to the present invention are known in the art and illustratively include benzyl alcohol, propylene glycol, water, art recognized equivalents and mixtures thereof.

Emollients are well known in the preparation of topical formulations, and a suitable choice for inclusion in a composition of the present invention will be recognized as such by one of skill in the art. Illustrative examples include hydrocarbons such as C8-C30 saturated or unsaturated fatty acids or alkyl esters of fatty acids, such as isopropyl myristate; sterols such as cholesterol and derivatives thereof; mixtures of emollients; and art recognized equivalents.

An antioxidant suitable for inclusion in an inventive composition includes any of those recognized in the art illustratively including ascorbic acid, BHT, BHA, a carotenoid, a tocopherol such as vitamin E acetate, a flavinoid, a glutathione. An antioxidant may be used independently or more than one may be used in an inventive composition.

A surfactant may be cationic, anionic, amphoteric, or nonionic or mixtures thereof. A non-ionic surfactant such as polysorbate 80 or an art recognized equivalent is preferred for use in an inventive topical treatment composition.

A composition according to the present invention may include a thickener or gelling agent such as Carbopol 980NF acrylic resin or an art recognized equivalent. A neutralizer may be added following dispersion of the thickener. For example, trolamine or an art recognized equivalent is useful in neutralizing an inventive composition.

Liposomes are a particularly preferred component of a topical treatment for acne. A composition including a liposomal gel preparation of the present invention, in addition to the aforementioned stability, has the advantages of containing skin-compatible ingredients, such as emollients, lipids and antioxidants, capable of soothing and promoting healing in damaged skin in an acne affected area without irritation. In addition, a liposomal gel preparation has the advantage of allowing delivery of hydrophilic, hydrophobic and amphipathic therapeutic agents since the preparation contains a lipid phase and an aqueous or continuous gel phase. The distribution of a therapeutic agent in a liposome will depend on numerous factors, such as, for example, characteristics of drug solubility, concentration of the drug, components of the liposome and the method of liposome preparation. Examples of known methods of liposome preparation are described in Liposomes: A Practical Approach, R.R.C. New, Editor, Oxford University Press, 1990, 1997.

Particularly preferred in the present invention is a liposomal gel preparation including an antibiotic in the aqueous or continuous phase and a retinoid in a lipid phase. Especially preferred is a liposomal gel preparation having clindamycin phosphate concentrated in the aqueous or continuous phase and tretinoin present in a lipid phase. While the compositions of this invention are described as having the retinoid contained in the lipid phase, it is to be understood that some relatively minor portion of the retinoid may partition into the aqueous phase over time. Such compositions will still manifest good long-term storage stability, and are within the scope of this invention. Preferably, compositions of the present invention have a majority of the retinoid contained in the lipid phase. In specifically preferred embodiments, at least 80% w/w of the retinoid is in the lipid phase, and in particular embodiments, 80-90% w/w of the retinoid is in the lipid phase. Disposing the retinoid in the lipid phase, in addition to enhancing storage stability, avoids problems of toxicity and the like which have heretofore limited the utility of retinoids.

The compositions of the present invention are used as topical agents for the treatment of acne and other skin conditions. Typically, they are applied to affected areas 1-3 times per day. It is generally preferred that the skin be cleaned with a mild cleanser prior to the application of the composition.

A method of making a liposomal gel preparation according to the present invention includes generating two phases, a lipid phase A including a retinoid and a gel dispersion phase B including an antibiotic. Phases A and B are mixed, resulting in the final preparation. Specific examples of inventive compositions and methods are described below.

### EXAMPLES

### Example 1

### An Example Formulation and Method of Preparation of a Retinoid/Antibiotic Acne Treatment Composition

A formulation according to the present invention is prepared in two phases, a lipid phase A and a gel dispersion phase B. The lipid phase preparation A is prepared by dissolving a retinoid in a solvent or a carrier. Preferred solvents are water-miscible materials such as alcohols, esters, ethers, and ketones. Such materials need not be infinitely soluble in water, but they should have some solubility, typically at least 10%. Following dissolution of the retinoid, oil phase materials including emollients, antioxidants and surfactants are added and mixed over gentle heat if necessary to achieve a uniform solution. Typically the material is heated to a temperature between 40° to 60° centigrade and mixed, as for example, with an anchor mixer at speeds of approximately 40 to 100 rpm. When the oil phase materials have melted, phospholipids are added to the mixture and gently agitated to achieve a solution without lumps. Again, gentle heating may be applied, usually in a range from 40° to 80° centigrade. Lipid phase preparation A is then mixed under light vacuum, for instance from about 10 to 30 inches of mercury (in-Hg) for a time sufficient to achieve a uniform preparation, typically ranging from 30 to 60 minutes using an anchor mixer at a speed ranging from 50 to 100 rpm. Preparation A is then cooled, for example, cooled to room temperature over a period of 90 minutes using an anchor mixer mixed on a water-circulating bath mixer under a low vacuum.

Preparation B is made by dissolving an antibiotic in a carrier. A thickener or gelling agent is slowly added into the antibiotic mixture and continuously stirred until a uniform and lump-free dispersion is achieved. Preparation B is then added to Preparation A and mixed. The resulting preparation is then mixed in a mixer/emulsifier and disperser under vacuum beginning as low as possible and climbing to a range of approximately 20 to 30 in-Hg for a time sufficient to achieve a uniform dispersion. After that time, the vacuum may be released and the product scraped from the agitators followed by a second mix step in a mixer/emulsifier and disperser under vacuum beginning as low as possible and climbing to a range of approximately 20 to 30 in-Hg for a time sufficient to achieve a smooth gel having a uniform dispersion. The resulting uniformly dispersed smooth gel has a liposome structure and very little or no crystalline structure when examined microscopically.

In other variants of this process, Preparation A is mixed with water prior to being mixed with Preparation B. This mixing creates the liposomal structure in Preparation A, and this liposomal structure is then blended with Preparation B.

### Example 2

### An Example Formulation of Tretinoin 0.025%/Clindamycin Phosphate 1.23% Acne Treatment Composition

### Procedure

Step 1. Weigh ingredients to be used. In this example, ingredients are included at percentages shown in Table 2 below.
Step 2. Start water circulation in a temperature-controlled homogenizer (Mokon) and set the temperature at 80° C.

### Lipid Phase Preparation

Step 3. Stir to dissolve tretinoin in benzyl alcohol and most of propylene glycol (part A) in a 1-L stainless steel container.

Stirring speed 5, solution temperature 50° C.
Step 4. Transfer oil phase materials: Isopropyl Myristate, Cholesterol, Vitamin E Acetate, Polysorbate 80 and BHT to a mix can. Mix to melt them (Anchor speed 75 rpm). When the oil phase materials are melted, add Phospholipon 90H to the mix can and mix to melt it.
Step 5. Heat purified water (part A) to 60° C.
Step 6. Transfer the tretinoin solution into the mix can while the anchor mixer is on. Rinse the SS container with Propylene Glycol. Mix for 5 minutes at 75 rpm.
Step 7. Add water (part A) to the mix can while the anchor mixer is on. Apply vacuum to the mix can at 15 in-Hg. Mix for 45 minutes.
   Anchor speed 75.2 rpm, vacuum level 15 in. Hg. Product temperature at the beginning 61.2° C. Product temperature at the end 52.1° C.
Step 8. Turn on the Mokon. Apply maximum vacuum to the mix can. Cool the product to room temperature for 90 minutes with the anchor mixer.

TABLE 1

| Mokon temperature | Product | Vacuum level |
|---|---|---|
| (° C.) | Temperature | (in-Hg) |
| 40 | 50.1 | 20 |
| 30 | 45.6 | 22 |
| 30 | 40.7 | 23 |
| 20 | 32.9 | 24 |
| 20 | 27.9 | 25 |

### Carbopol Dispersion Preparation

Step 9. Set up the Lightnin mixer. Add most of the water (part B) and propylene glycol (part B) into a stainless steel container. Start mixing at 600 rpm.
Step 10. Add clindamycin phosphate into the above container. Rinse the container with water. Dissolve clindamycin phosphate.
Step 11. Add Carbopol slowly into the stainless steel container. Continue to stir until a uniform and lump-free dispersion is achieved.
Step 12. Stop the vacuum. Transfer Carbopol dispersion to the mix can while the anchor mixer is on, rinse the container with rinse water.
Step 13. Mix for 10 minutes using the anchor mixer at 50 rpm.
Step 14. Add Trolamine. Rinse the container using the rinse water.
Step 15. Apply vacuum. Mix with mixer/emulsifier at 1700 rpm and disperser at 900 rpm for 5 minutes with the anchor mixer running at 50 rpm.
Step 16. Apply maximum vacuum. Continue mixing with mixer/emulsifier at 1700 rpm and disperser at 900 rpm for 5 minutes with the anchor mixer running at 50 rpm.
Step 17. Stop the mixer/emulsifier and disperser and release the vacuum. Stop the anchor. Scrape the product from the agitators.
Step 18. Start the anchor mixer and apply maximum vacuum. Mix with mixer/emulsifier at 1700 rpm and disperser at 900 rpm for 10 minutes with the anchor mixer running at 50 rpm.
Step 19. Stop the anchor mixer, mixer/emulsifier and disperser and release the vacuum.
Step 20. Scrape the product from the agitators. Take samples in order to evaluate the final product visually, chemically, microscopically and microbiologically. Measure the yield, pH and viscosity and assay for active ingredients and benzyl alcohol

### Results

Opaque, slightly yellow gel. Smooth. Uniformly dispersed. A lot of liposome structures. No crystals presented. Passed cycle study.

TABLE 2
Example Formulation of Tretinoin/Clindamycin
Phosphate Acne Treatment Composition

| Ingredient | % w/w |
|---|---|
| Benzyl Alcohol, NF | 2.0 |
| Isopropyl Myristate, NF | 8.0 |
| Cholesterol, USP | 0.3 |
| Tretinoin, USP | 0.025 |
| Propylene Glycol USP (part A) | 10.0 |
| Vitamin E Acetate, USP | 0.3 |
| Polysorbate 80, NF | 0.75 |
| Phospholipon 90H | 3.0 |
| BHT, NF | 0.1 |
| Purified Water, USP (part A) | 28.0 |
| Carbopol 980, NF | 0.4 |
| Clindamycin Phosphate, USP | 1.23 |
| Trolamine, NF | 0.25 |
| Propylene Glycol, USP (part B) | 10.0 |
| Purified Water, USP (part B) | 35.55 |

### EXPERIMENTAL EVALUATION

The efficacy of the present invention was evaluated in an experimental series directed to measuring the stability of compositions of a retinoid (tretinoin) and an antibiotic (clindamycin phosphate). Specifically, a first composition was prepared in accord with the present invention as per the methodology and ingredients described in Example 2 hereinabove. This composition included, on a weight basis, 1.23% of clindamycin phosphate and 0.025% of tretinoin. In accord with the present invention, and as per the methodology of Example 2, the composition was formulated to include a liposomal vehicle comprising a lipid phase disposed in an aqueous phase, wherein the clindamycin phosphate antibiotic was disposed in the aqueous phase and the tretinoin was disposed in the lipid phase. A generally similar composition, composition II, was prepared utilizing all of the ingredients of Example 2 hereinabove, except for the Phospholipon 90H phosphatidyl choline. Because of the elimination of the phospholipid material, composition II could not form any liposomes, and hence did not provide any structure which isolated the clindamycin phosphate from the tretinoin. As such, composition II, while very similar to composition I with regard to its ingredients, lacked the inventive microstructure of the present invention. As such, composition II is typical of prior art preparations.

Both of the compositions were subjected to accelerated aging conditions wherein they were stored at a temperature of 40°±2° C. at 75% relative humidity ±5% relative humidity. These accelerated aging conditions are typically employed in the pharmaceutical industry to evaluate the long term storage stability of pharmaceutical preparations. Over the course of the study, the appearance of the two compositions and their concentrations of clindamycin, tretinoin, and benzyl alcohol were evaluated, with regard to established standards for the product. Data from the evaluation of composition. I is summarized in table 3, and data from composition II is summarized in Table 4.

With regard to appearance, standards require that the product visually appear as an opaque, yellowish-white gel. Both composition I and composition II presented this appearance at the start of the evaluation; however, at the one-month point composition II no longer met this appearance standard, while composition I continued to satisfactorily meet the standard for the duration of the six-month evaluation. With regard to the content of the clindamycin, tretinoin, and benzyl alcohol, the standards require that the concentration of these materials in the composition fall in the range of 90-110% of the nominally stated amount. In evaluating compositional stability, two or three samples were analyzed for each composition at the start of the study, and at subsequent one month, two month, three month, and six month intervals. As will be seen from the data, composition I maintained a satisfactory clindamycin level through the third-month evaluation and fell below standards at the six-month level. In contrast, composition II failed to show a satisfactory clindamycin level at the three-month evaluation and no further testing was carried out at six months. With regard to tretinoin and benzyl alcohol levels, both samples showed satisfactory levels at the three-month point, although it is to be noted that the levels of tretinoin in composition II were slightly lower at this point, and the tretinoin levels in composition I were satisfactory at the six-month point. Both compositions showed stable benzyl alcohol levels.

TABLE 3

| Test | Specification | Initial | 1 Month | 2 Months | 3 Months | 6 Months |
|---|---|---|---|---|---|---|
| Appearance | opaque, | satisfactory | satisfactory | satisfactory | satisfactory | satisfactory |
| | yellowish-white | | | | | |
| | gel satisfactory | | | | | |
| Clindamycin | 90% claim | 101.1 | 99.3 | 95.8 | 93.2 | 86.6 |
| assay | 110% claim | 101.3 | 98.9 | 95.7 | 93.1 | 86.9 |
| | 1% w/w | 101.3 | 98.7 | 95.4 | 92.9 | 87.4 |
| Tretinoin | 90% claim | 100.6 | 100.0 | 98.0 | 99.6 | 104.4 |
| assay | 110% claim | 96.4 | 99.6 | 97.6 | 98.8 | 103.6 |
| | 0.025% w/w | 99.0 | 100.0 | 97.2 | 98.4 | 102.4 |
| Benzyl alcohol | 90% claim | 101 | 102 | 102 | 101 | 100 |
| assay for CLN | 110% claim | 102 | 102 | 102 | 102 | 100 |
| | 2% w/w | 101 | 102 | 102 | 101 | 100 |

TABLE 4

| Test | Specification | Initial | 1 Month | 2 Months | 3 Months | 6 Months |
|---|---|---|---|---|---|---|
| Appearance | opaque, | satisfactory | unsatisfactory | Un | Un | — |
| | yellowish- | | | satisfactory | satisfactory | |
| | white gel | | | | | |
| | satisfactory | | | | | |
| Clindamycin | 90% claim | 103.6 | 97.4 | 92.2 | 89.7 | — |
| assay | 110% claim | 103.4 | 97.3 | 92.1 | 89.4 | — |
| | 1.2% w/w | | 97.3 | 92.4 | 89.2 | — |
| Tretinoin | 90% claim | 100.4 | 97.2 | 97.6 | 97.6 | — |
| assay | 110% claim | 100.4 | 97.2 | 98.0 | 98.0 | — |
| | 0.025% w/w | | 97.6 | 98.4 | 97.6 | — |
| Benzyl | 90% claim | 101 | 101 | 100 | 101 | — |
| alcohol assay | 110% claim | 101 | 101 | 100 | 101 | — |
| for CLN | 2% w/w | | 100 | 100 | 101 | — |

This experimental series demonstrates that compositions of the present invention, in which an antibiotic composition and a retinoid component are isolated from one another, provide significantly enhanced stability as compared to very similar compositions in which no isolation of the ingredients is maintained. This enhanced stability is significant both commercially and therapeutically. As detailed above, the prior art does not show or suggest any topical compositions of retinoids and antibiotics wherein liposomal structures are used to isolate the component active ingredients. Furthermore, the prior art does not show or suggest to one of skill in the art that benefits of stability would be achieved through the use of such preparations. The results of the present invention are significant, novel, and unanticipated.

A process for treating acne is provided by the present invention. An inventive process includes the steps of providing a composition including a liposomal gel preparation of an antibiotic and a retinoid and applying the composition to an area of skin affected by acne in a subject having acne.

While the foregoing describes liposomal compositions in which the lipid phase is discontinuous and the aqueous phase is continuous, reverse structures are known in the art, and they may also be employed in the present invention. In such structures, the retinoid will be in a continuous, lipid phase and the antibiotic in a discontinuous aqueous phase.

One skilled in the art will readily appreciate that the present invention is well adapted to carry out the objects and obtain the ends and advantages mentioned, as well as those inherent therein. The present methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

1. A stabilized, topical composition for the treatment of skin conditions, said composition comprising:
a liposomal vehicle comprising a lipid phase disposed in an aqueous phase;
an antibiotic disposed in said aqueous phase; and
a retinoid disposed in said lipid phase.

2. The composition of claim 1, wherein said retinoid is present, on a weight basis, in a range of 0.01-1.0%.

3. The composition of claim 1, wherein said antibiotic is present, on a weight basis, in a range of 0.5-10%.

4. The composition of claim 1, wherein said retinoid comprises tretinoin.

5. The composition of claim 4, wherein said tretinoin is present, on a weight basis, in the range of 0.01-0.75%.

6. The composition of claim 4, wherein said tretinoin is present, on a weight basis, in the range of 0.02-0.05%.

7. The composition of claim 1, wherein said antibiotic comprises a lincosamide antibiotic.

8. The composition of claim 7, wherein said lincosamide antibiotic comprises clindamycin.

9. The composition of claim 8, wherein said clindamycin comprises clindamycin phosphate.

10. The composition of claim 8, wherein said clindamycin is present, as the clindamycin base, on a weight basis in the range of 0.75-2%.

11. The composition of claim 1, wherein at least 80% of said retinoid is disposed in said lipid phase.

12. The composition of claim 1, further including an ancillary ingredient selected from the group consisting of: emollients, antioxidants, surfactants, thickeners, gelling agents, pH control agents, coloring agents, fragrances, solvents, carriers, dispersion agents, and combinations thereof.

13. A method for manufacturing a stabilized, topical composition for he treatment of skin conditions, said method comprising the steps of:
preparing a lipid phase by dissolving a retinoid and a phospholipid in a water miscible solvent to produce a lipid solution; adding water to said lipid solution; and mixing said water and said lipid solution so as to produce a liposomal material in which said retinoid is disposed within the lipid phase of said liposomal material;
preparing an aqueous phase by dissolving an antibiotic and a thickening agent in an aqueous based solvent system so as to produce a thickened, aqueous based solution of said antibiotic; and
mixing said liposomal material and said aqueous based solution of said antibiotic.

14. The method of claim 13, wherein the step of preparing said lipid phase comprises the further step of dissolving an additional material in said water miscible solvent, said additional material being selected from the group consisting of: cholesterol, antioxidants, alkyl esters of fatty acids, and combinations thereof.

15. The method of claim 13, wherein said water miscible solvent is selected from the group consisting of: alcohols, glycols, ketones, esters, and combinations thereof.

16. The method of claim 13, wherein said water miscible solvent comprises a mixture of benzyl alcohol and propylene glycol.

17. The method of claim 13, wherein the step of mixing said aqueous based solution of said antibiotic and said liposomal material comprises emulsifying said aqueous based solution and said liposomal material.

18. A method for treating a patient having acne, said method comprising applying to said patient's skin a composition comprising:
a liposomal vehicle comprising a discontinuous, lipid phase dispersed in a continuous, aqueous phase;
an antibiotic disposed in said aqueous phase; and
a retinoid disposed in said lipid phase.

19. A topical composition for the treatment of acne, said composition comprising:
a liposomal vehicle comprising a discontinuous, lipid phase disposed in a continuous, aqueous phase;
clindamycin, in a weight amount of 0.5-10%, as the clindamycin base, disposed in said aqueous phase; and
0.01-1.0%, on a weight basis, of a retinoid disposed in said lipid phase.

20. The composition of claim 18, wherein said retinoid comprises tretinoin, and is present in said lipid phase in a weight range of 0.01-0.75%.
